(19) United States
(12) Patent Application Publication
(10) Pub. No.: US 2008/0228117 A1
(43) Pub. Date: Sep. 18, 2008

(54) Apparatus for the treatment of arm disorders and the methods of using same

(76) Inventor: Donald O. Fareed, Santa Barbara, CA (US)

Correspondence Address: JAMES E. BRUNTON, ESQ., P. O. BOX 29000, GLENDALE, CA, 91209, US

(21) Appl. No.: 11/724,807

(22) Filed: Mar. 15, 2007

(51) Int. Cl. A61F 5/00

(52) U.S. Cl. 602/5; 602/20

(57) ABSTRACT

A method treating the symptoms of tendonitis, arthritis, capsulities, lymphedema and ganglion cysts in the arm of a user which involves the steps of placing a pressure imparting device around selected portions of the user's arm and applying controlled pressure to the discrete areas of the skin directly overlying the selected portions. The pressure imparting device includes two discrete compression plates that are placed around selected portions of the user's arm and are drawn toward one another to apply bipolar transaxial compression of equal magnitude simultaneously to discrete areas of the skin directly overlying the selected portions.

### BACKGROUND OF THE INVENTION

1. Field of the Invention

The present invention relates generally to methods and apparatus for treating disorders in the arm of a patient. More particularly, the invention concerns methods and apparatus for treating the symptoms of biceps/triceps tendonitis and lymphedema in a patient through the strategic placement about the arm of the patient of novel pressure imparting devices of unique design.

2. Discussion of the Prior Art

The prior art is replete with various types of devices that can be positioned about the arms, wrists, legs, ankles and feet of a user that are touted to provide relief from numerous types of physical problems. Most of these devices provide some support to a particular member, but few have any therapeutic value for treating disorders associated with injuries and diseases involving circulation of blood to and from the member.

A number of highly successful devices, which do have proven therapeutic value, have been developed by the present inventor. One such device is described in U.S. Pat. No. 5,152,302 entitled Forearm Transaxial Compression Band. This patent, which issued Oct. 6, 1992, describes an adjustable strap or band adapted to be circumferentially fitted around the forearm of a patient to alleviate the symptoms of tennis elbow. The strap is a generally band-shaped device having opposing inwardly protruding means on its forearm contacting surface to direct transaxial compression against the radial extensor, separator complex and flexor muscles when the band is circumferentially tensioned and fastened in place around the forearm. In its preferred form, the band applies an adjustable pressure principally upon the extensor, supinator and flexor wads permitting unimpeded blood circulation along all other portions of the forearm. The two opposing means for applying transaxial compression are limited in their circumferential extent to those areas of the band immediately overlying the extensor and flexor muscle mass.

Another highly successful therapeutic method and apparatus invented by the present inventor is described in U.S. Pat. No. 5,295,951 entitled Device and Method for Treating Tennis Elbow. This patent, which issued Mar. 22, 1994, discloses a method for treating the symptoms of tennis elbow. One form of this method comprises the steps of presenting a generally circular device that comprises a means for concentrating a radially compressive force onto the forearm, the means for concentrating the force are two surfaces projecting inwardly from the circular device and located at two distinct positions that are transaxially opposed to each other; placing the device around the forearm to encircle at least a portion of the forearm; and applying bipolar transaxial compression of equal magnitude simultaneously to discrete areas of the skin directly overlying the flexor and the extensor muscles of the forearm without substantial compression of other skin of the forearm.

In the course of extensive use of the device described in the patents identified in the preceding paragraphs, the present inventor has discovered that, surprisingly, the device described in U.S. Pat. No. 5,152,302 relieves and/or resolves intra-articular inflammation, arthritis and synovial impingement from within the elbow joint, which action is independent of its use with tennis elbow and tendonitis. The inventor has also found that patients wearing the device frequently observe improvement of their arthritis. Additionally, it has been observed that use of the device unexpectedly results in the disappearance of ganglion cysts that typically occur in the wrist of a patient.

Further, it has been observed that people with chronic bilateral lymphedema following mastectomy experience immediate resolution of the lymphedema in the arm to which the device was applied. It has also been determined that when the device is used in conjunction with repetitive grip contraction, the beneficial action of the device dramatically accelerates.

As a result of the extensive use of the device described in the patents identified in the preceding paragraphs, the present inventor has also developed other highly novel pressure imparting devices, including a novel elbow compressor the character of which will be described in detail in the paragraphs that follow.

### SUMMARY OF THE INVENTION

By way of summary, one form of the method of the invention for treating the symptoms of tendonitis, arthritis, capsulities, lymphedema and ganglion cysts in the arm of a user involves the steps of placing a pressure imparting device around selected portions of the user's arm and applying controlled pressure to the discrete areas of the skin directly overlying the selected portions. More particularly, one form of the method of the invention for treating the symptoms of tendonitis, arthritis, capsulities, lymphedema and ganglion cysts in the arm of a user involves the steps of placing a generally circular device having two discrete compression plates around selected portions of the user's arm and drawing the compression plates toward one another to apply bipolar transaxial compression of equal magnitude simultaneously to discrete areas of the skin directly overlying the selected portions.

With the foregoing in mind, it is an object of this invention to provide a method for treating the symptoms of a biceps/triceps tendonitis and lymphedema in the arm of a user through use of a compression band placed around the biceps/triceps portion of the user's arm.

Another object of the invention is to provide a treatment method that involves the use of a compression band of the character described in the preceding paragraph with means on the inner surface thereof for selectively applying transaxial counter-compression against the biceps/triceps muscles where the counter-compression means can be easily adjusted by the user while in use.

Another object of the invention is to provide a method for treating the symptoms of arthritis, tendonitis, capsulities and ganglion cysts in the arm of a user through the use of a compression band of the character described that is placed around the elbow portion of the user's arm.

Another object of the invention is to provide a method, as described in the previous paragraph, which uses a compression band that includes means on the inner surface thereof for selectively applying transaxial counter-compression against the elbow portion.

Another object of the invention is to provide a novel apparatus for treating the symptoms of arthritis, tendonitis, capsulities and ganglion cysts in the arm of a user.

Another object of the invention to provide a novel elbow compression apparatus for treating and preventing the symptoms of arthritis, capsulitis, synovitis, biceps-triceps tendonitis, carpal tunnel syndrome, lymphedema, ganglion cyst, trigger finger and like maladies.

### BRIEF DESCRIPTION OF THE DRAWINGS

FIG. 1 is a generally perspective view illustrating one form of the method of the present invention for treating the symptoms of a biceps/triceps tendonitis and lymphedema in the arm of a user through use of a compression band placed around the biceps/triceps portion of the user's arm.

FIG. 2 is a side-elevational view further illustrating the method of one form of the invention for treating the symptoms of a biceps/triceps tendonitis and lymphedema in the arm of a user.

FIG. 3 is a generally perspective view illustrating another form of the method of the invention for treating the symptoms of arthritis, tendonitis, capsulities and ganglion cysts in the arm of a user through the use of a generally circular compression band placed around the elbow portion of the user's arm.

FIG. 4 is a cross-sectional view taken along lines 4-4 of FIG. 2.

FIG. 5 is a cross-sectional view taken along lines 5-5 of FIG. 3.

FIG. 6 is a generally perspective view illustrating one form of the apparatus of the invention for treating dysfunctions of the arm of a patient by positioning the apparatus around the elbow portion of the patient's arm in the manner shown in the drawing.

FIG. 6A is an enlarged, generally perspective view of the apparatus of the invention shown in FIG. 6.

FIG. 6B is a generally perspective view, similar to FIG. 6A, but showing the elbow-encircling strap having been pivoted from the first position shown in FIG. 6A into a second, lowered position.

FIG. 7 is a generally perspective view illustrating the apparatus of the invention as it appears in FIG. 6B when positioned around the upper forearm of the patient.

FIG. 8 is a generally perspective view illustrating an alternate form of the apparatus of the invention for treating dysfunctions of the arm of a patient by positioning the apparatus around the elbow portion of the patient's arm in the manner shown in the drawing.

FIG. 9 is an enlarged, generally perspective view of yet another form of the apparatus of the invention for treating dysfunctions of the arm of a patient.

FIG. 10 is a generally perspective view of still another form of the invention as it appears when positioned about the elbow of the patient.

FIG. 11 is an enlarged, generally perspective view of the embodiment of the invention shown in FIG. 8 of the drawings.

### DESCRIPTION OF THE INVENTION

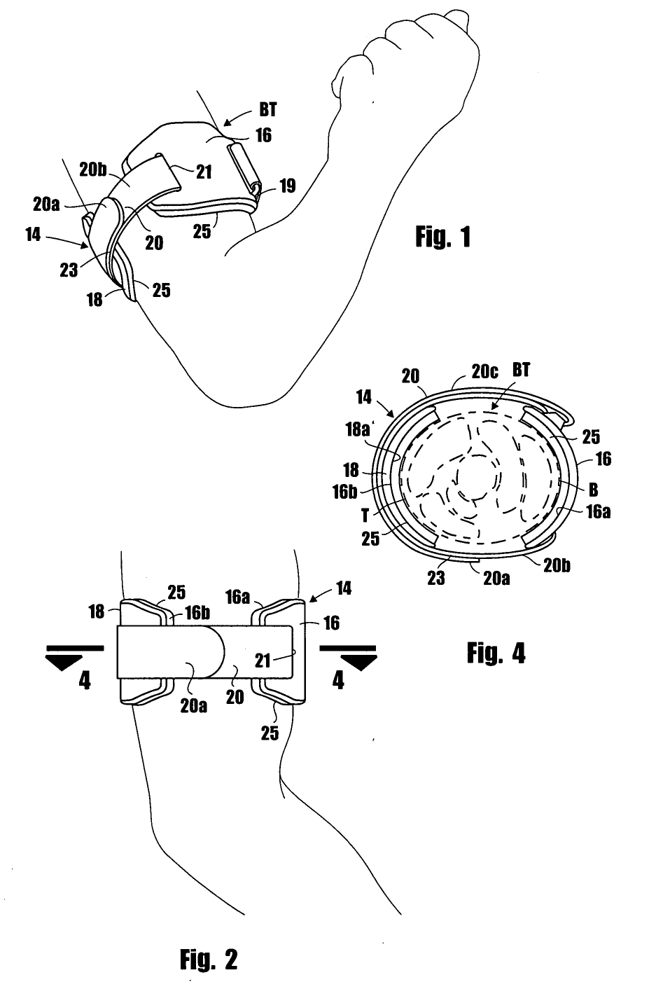

Referring to the drawings, and particularly to FIGS. 1, 2 and 4, the accomplishment of one form of the method of the invention is there illustrated. This method is directed toward the treatment of the symptoms of biceps/triceps tendonitis and lymphedema in a user through the use of a generally circular compression device 14 having two discrete compression plates 16 and 18 that are linked to one another by a compression inducing member here shown as an elongated strap 20 in a manner such that they project inwardly from the circular device. In FIGS. 2 and 4 of the drawings strap 20 is shown encircling the biceps/triceps portion “B-T” of the user's arm.

Tendonitis, which is one of the symptoms treated by the method of the invention, occurs when the tendon(s) around a particular joint have become severely inflamed from overuse, micro-injury, etc. Biceps/triceps tendonitis occurs when the tendon(s) in the area of the biceps/triceps have become severely inflamed. It is this highly unpleasant symptom, among others, that the method of the present invention seeks to alleviate.

As illustrated in FIGS. 1 and 4, when in use, the strap 20 is positioned about the upper arm so that the inelastic compression plates 16 and 18, which have concave inner skin-contacting surfaces 16a and 16b, strategically overlie the bicep area “B” and the triceps area “T” respectively to enable effective transaxial counter-compression of these areas (FIG. 4). The strap 20 passes through spaced-apart slots 21 formed in the compression plates and free end of the strap 20a is provided with connector means, such as a material 23 sold under the name and style VELCRO, so that the length of strap 20 can be circumferentially adjusted to optimize transaxial compression. The presence of the strap segments 20b and 20c, which overlie the non-target areas insure unrestricted circulation of the blood. The skin contacting surface of the compression plates are conveniently lined with closed-cell foam 25 for comfort.

For a more detailed description of the details of the construction of the compression device 14, reference should be made to U.S. Pat. No. 5,152,302, which patent is hereby incorporated by reference as though fully set forth herein.

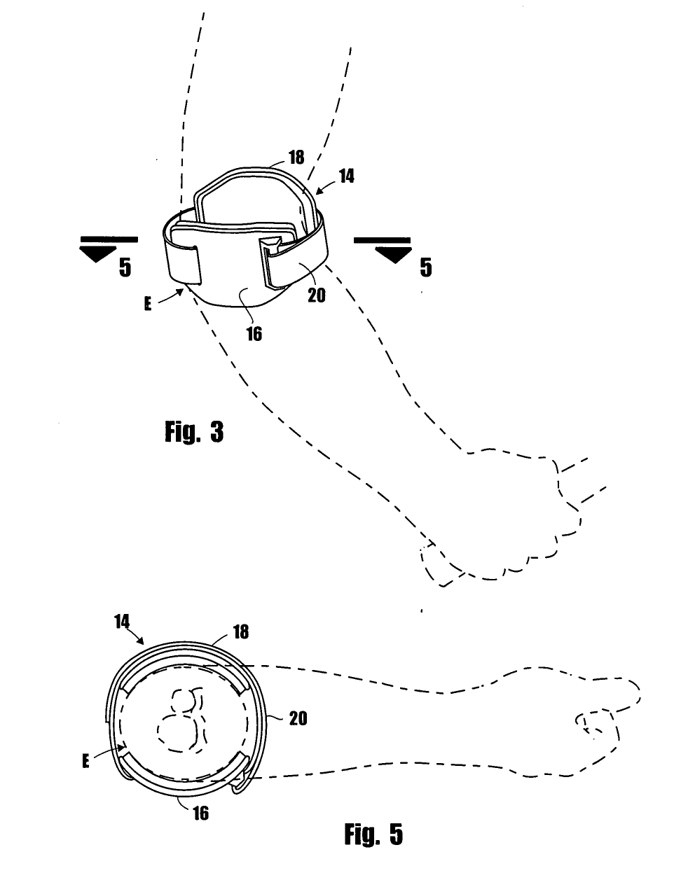

Referring next to FIGS. 3 and 5, the accomplishment of an alternate form of the method of the invention is there illustrated. The method of this form of the invention is directed toward the treatment of the symptoms of arthritis, tendonitis, capsulities and ganglion cysts in the arm of a user through the use of the previously identified compression device 14 which is identical in construction to that previously described.

This alternate method of the invention comprises the steps of first placing the device 14 around the elbow portion “E” of the user's arm in the manner shown in FIGS. 3 and 5. In this position, the device encircles the elbow portion so that the discrete compression plates 16 and 18 overlie the elbow portion in the manner best seen in FIG. 5. This done, using the strap 20, the compression plates are drawn toward one another to apply bipolar compression to the elbow portion.

Arthritis, which is one of the symptoms treated by this alternate method of the invention, is frequently accompanied by joint pain and occurs when one or more joints have become inflamed and sometimes overproduces fibrous tissue to the area of the inflammation. Capsulitis, which is another of the symptoms treated by this alternate method of the invention, means that the connective tissue which surrounds the bony joints of the body has become inflamed. The cause is usually muscle weakness. The word “ganglion” means “knot” and is used to describe the knot-like mass or lump of cells that forms below the surface of the skin. The ganglion cysts, which are treated by this alternate method of the invention, comprise the knot-like mass or gel-filled sack of cells that have formed in the forearm and wrist area of the patient distant from the site of the application.

As previously discussed, in the course of extensive use of the device described in the preceding paragraphs, the inventor discovered that, quite unexpectedly, the device relieves and/or resolves the foregoing forearm, wrist and hand symptoms independent of its use with tennis elbow and tendonitis. The inventor has also observed that use of the device in the manner described herein surprisingly results in the disappearance of ganglion cysts that typically occur in the wrist of a patient.

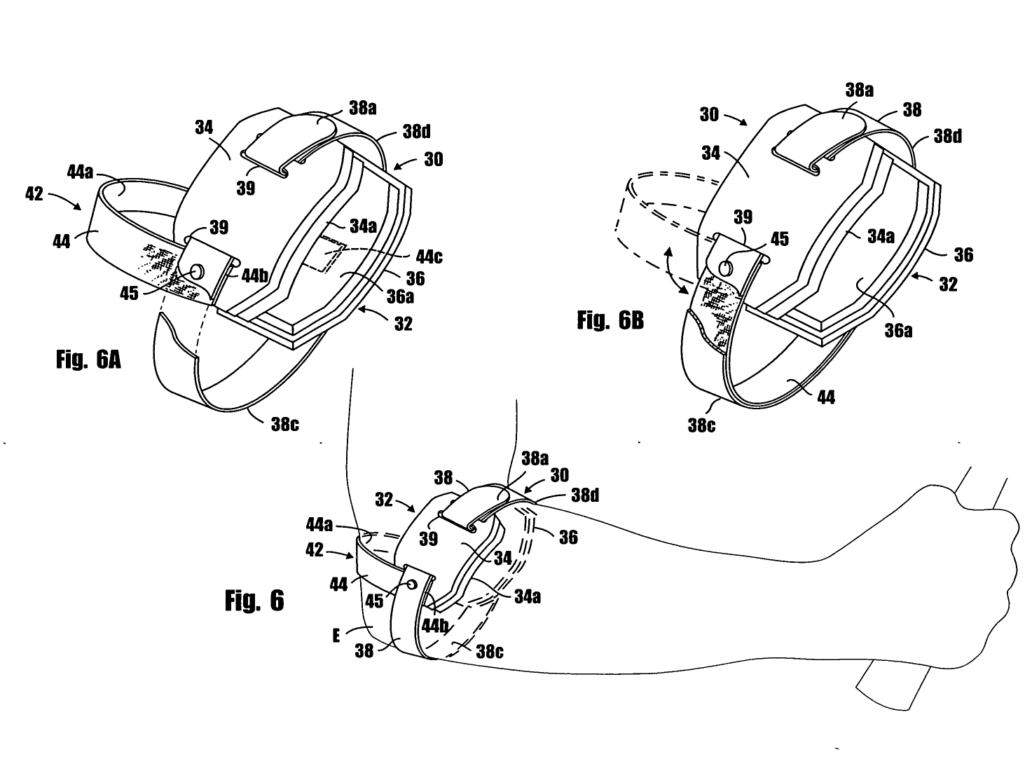

Referring next to FIGS. 6, 6A and 6B, an alternate form of the apparatus and the method of the invention is there illustrated. The apparatus, the details of which will presently be described, is somewhat similar to the apparatus illustrated in FIGS. 1 through 5 of the drawings. The method of this alternate form of the invention is also similar to that described in connection with FIGS. 1 through 5 and is directed toward the treatment of the symptoms of arthritis, tendonitis, capsulities, carpal tunnel syndrome, lympedema, trigger finger and ganglion cysts in the arm of a user through the use of a modification of the previously identified compression device 14.

This modified device, which is generally designated by the numeral 30, comprises a first portion 32 which includes a first forearm-engaging member 34 and a circumferentially spaced forearm-engaging second member 36 that is linked to first 34 by means of a compression inducing member here provided as an adjustable, flexible strap 38. Flexible strap 38 is constructed and arranged as to cause the spaced-apart arm-engaging members to exert pressure on selected areas of the forearm without substantially compressing other non-target areas and tissues. More particularly, as illustrated in FIG. 6, when in use, the strap 38 is positioned about the patient's arm proximate the elbow so that the inelastic compression members 34 and 36, which have concave inner skin-contacting surfaces 34a and 36a, strategically overlie the forearm so as to enable effective transaxial counter-compression of the contact areas. The strap 38 passes through spaced-apart slots 39 formed in the compression members and free end 38a of the strap 38 is provided with connector means, such as a suitable material sold under the name and style VELCRO, so that the length of strap 38 can be circumferentially adjusted to optimize transaxial compression. The presence of the strap segments 38c and 38d, which overlie the non-target areas insure unrestricted circulation of the blood.

The modified device, 30 of this latest form of the invention also comprises a second portion which is generally designated by the numeral 42. Second portion 42, which is interconnected with first portion 32, here comprises a generally U-shaped flexible strap 44 having a bite portion 44a for engaging the patient's arm proximate the triceps and first and second end portions 44b and 44c that are pivotally connected to first portion 32 by means of suitable pivot pins 45 (FIGS. 6 and 6A). With this construction, second flexible strap 44 can be moved between the first elbow-encircling position shown in FIG. 6 (and shown by the phantom lines in FIG. 6B) and a second position shown in FIG. 6B wherein the bite portion 44a of the strap is in close proximity with said first flexible strap, 38.

Turning particularly to FIG. 6, an alternate method of the invention for treating arthritis, capsulitis, synovitis and like maladies relating to the arm of the patient is there shown. This latest method of the invention comprises the steps of first placing the modified device 30 around the elbow portion “E” of the user's arm in the manner shown in FIG. 6. In this position, the device encircles the elbow portion so that the discrete compression members 34 and 36 overlie the forearm portion of the patient and the U-shaped flexible strap 44 extends around the elbow so that a bite portion 44a thereof engages the rear portion of the arm at a location just above the elbow portion “E”. With the device in this position the strap 38 is tightened so that the compression members are drawn toward one another to apply a uniform bipolar compression to the forearm.

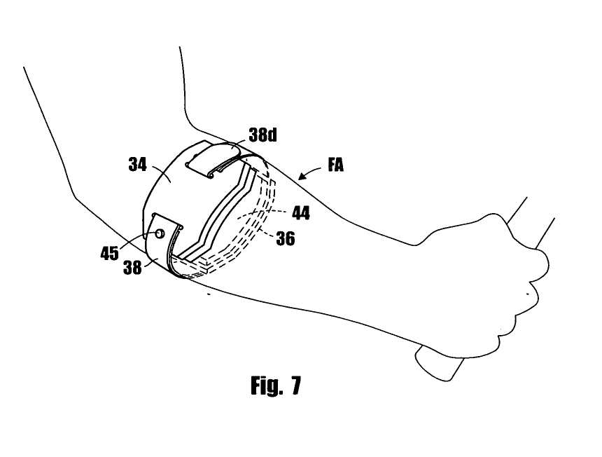

With the device of the invention in the configuration shown in FIG. 6B it can be used in the manner shown in FIG. 7 of the drawings to accomplish yet another method of the invention. This alternate form of the method of the invention is directed to the treatment of disorders related directly and indirectly to the patient's forearm as, for example, carpal tunnel syndrome, arthritis, tendonitis, capsulities, lympaedema, trigger finger and ganglion cysts in the arm of a user, including symptoms related to the hand and fingers of the patient. This latest method of the invention comprises the steps of first placing the modified device 30 around the forearm portion “FA” of the patient in the manner shown in FIG. 7. In this position, the device encircles the forearm portion so that the discrete compression members 34 and 36 overlie the forearm with the U-shaped flexible strap 44 having been pivoted into the second position in close proximity with compression member 36. With the device in this position the strap 38 is tightened so that the compression members are drawn toward one another to apply a uniform bipolar compression to the forearm portion.

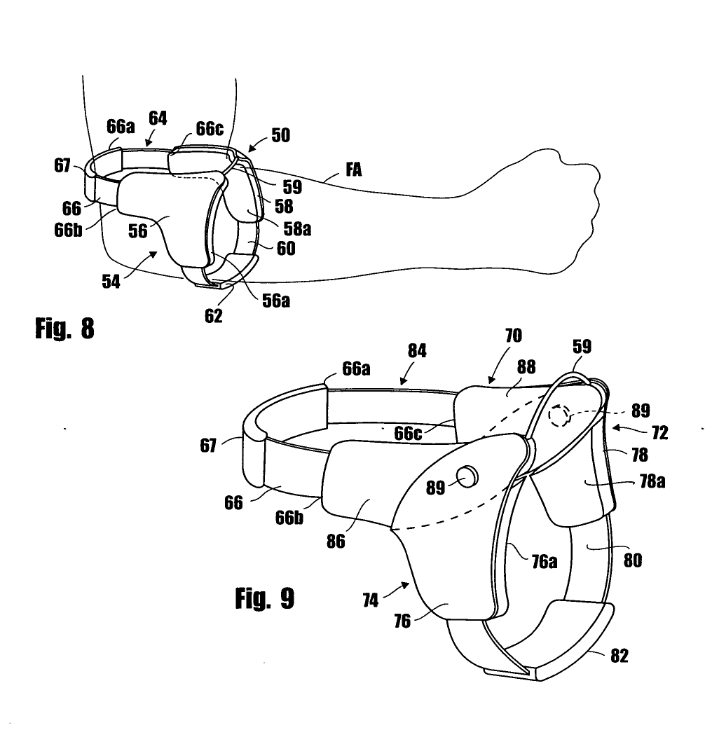

Turning now to FIGS. 8 and 11, still another form of the method and apparatus of the invention is there illustrated. The compression device of this embodiment of the invention, which is generally designated by the numeral 50, is also somewhat similar to that illustrated in FIGS. 6, 6A and 6B of the drawings. The method of this alternate form of the invention is also similar to that described in connection with FIGS. 6 and 7 and is directed toward the treatment of arthritis, tendonitis, capsulities, carpal tunnel syndrome, lympedema, trigger finger and ganglion cysts in areas directly and indirectly related to the arm of the patient.

As best seen in FIG. 11 of the drawings, the compression device 50 here comprises a first portion 52 which includes a first forearm-engaging assembly generally designated by the numeral 54. Assembly 54 includes first and second spaced-apart side members 56 and 58 that are interconnected by a compression inducing connector member shown here as a polymer cord 59. Assembly 54 also includes a flexible strap 60 that is interconnected with and depends downwardly from the side members 56 and 58. Also comprising a part of first forearm-engaging assembly 54 is a compression pad 62 that is carried by flexible strap 60 in the manner shown in the drawings. Polymer cord 59 is constructed and arranged so as to cause the spaced-apart arm-engaging side members 56 and 58 to exert an inward pressure on selected areas of the forearm without substantially compressing other non-target areas and tissues. More particularly, as illustrated in FIG. 8, when the device is in use, the side members 56 and 58 are positioned about the patient's forearm proximate the elbow so that the inelastic side members 56 and 58, which have concave inner skin-contacting surfaces 56a and 58a respectively, strategically overlie the forearm so as to enable effective transaxial counter-compression of the contact areas.

The device, 50 of this latest form of the invention also includes a second portion which is generally designated by the numeral 64. Second portion 64 here comprises a generally U-shaped flexible strap 66 having a bite portion 66a and first and second end portions 66b and 66c. As illustrated in FIG. 11 of the drawings, end portions 66b and 66c are interconnected with side members 56 and 58 by any suitable means such as sewing. For certain applications, it is desirable that the second strap 44 be constructed from a stretchable material. Also forming a part of second portion 64 is a compression member 67 which is carried by the bite portion 66a of strap 66 in the manner shown in FIG. 11 of the drawings.

Device 50 can be used in the manner illustrated in FIG. 8 to accomplish still another method of the invention that is directed to the treatment of disorders related directly and indirectly to the patient's forearm as, for example, treatment of the symptoms of arthritis, tendonitis, capsulities, carpal tunnel syndrome, lymphedema, trigger finger and ganglion cysts in the arm of a user, including symptoms related to the hand and fingers of the patient. This latest method of the invention comprises the step of placing the device 50 around the forearm portion “FA” of the patient in the manner shown in FIG. 8. In this position, the first portion 54 of the device encircles the forearm portion so that the discrete side compression members 56 and 58 compressively engage the sides of the forearm proximate the elbow and so that the pad 62 compressively engages the underside of the forearm. As illustrated in FIG. 8, with the device correctly in position, the generally U-shaped flexible strap 66 of the second portion 64 of the device encircles the arm at a location just above the elbow so that the pad 67 compressively engages the arm at this location.

Referring next to FIG. 9, still another form of the apparatus of the invention is there illustrated. The compression device of this embodiment of the invention, which is generally designated by the numeral 70, is quite similar to that illustrated in FIGS. 8 and 11 of the drawings and like numerals are used in FIG. 9 to identify like components. The primary difference between this latest form of the invention and that shown in FIGS. 8 and 11 resides in the fact that the second portion of the device is pivotally interconnected with the first portion. Compression device 70 here comprises a first portion 72 which includes a first forearm-engaging assembly generally designated by the numeral 74. Assembly 74 includes first and second spaced-apart side members 76 and 78 that are interconnected by an adjustable connector member here shown as a polymer cord 59. Assembly 74 also includes a flexible strap 80 that is interconnected with and depends downwardly from the side members 76 and 78. Also comprising a part of first forearm-engaging assembly 74 is a compression pad 82 that is carried by flexible strap 80 in the manner shown in the drawings. As before, polymer cord 59 is constructed and arranged so as to cause the spaced-apart arm-engaging side members 76 and 78 to exert an inward pressure on selected areas of the forearm without substantially compressing other non-target areas and tissues. More particularly, as illustrated in FIG. 9, when the device is in use, the side members 76 and 78 are positioned about the patient's forearm proximate the elbow so that the inelastic side members 76 and 78, which have concave inner skin-contacting surfaces 76a and 78a respectively, strategically overlie the forearm so as to enable effective transaxial counter-compression of the contact areas.

The device, 70 of this latest form of the invention, also includes a second portion which is generally designated by the numeral 84. Second portion 84 here comprises a generally U-shaped flexible strap 66 having a bite portion 66a and first and second end portions 66b and 66c. As illustrated in FIG. 9 of the drawings, end portions 66b and 66c are interconnected with the connector portions 86 and 88, which here comprise a part of second portion 84. Also forming a part of second portion 84 is a compression band 67 which is carried by the bite portion 66a of strap 66 in the manner shown in FIG. 9 of the drawings. Connector portions 86 and 88 are pivotally interconnected with side members 76 and 78 by any suitable means, such as pivot pins 89. With this construction, second portion 84 can be pivoted relative to first portion 72 as may be required to comfortably fit the patient.

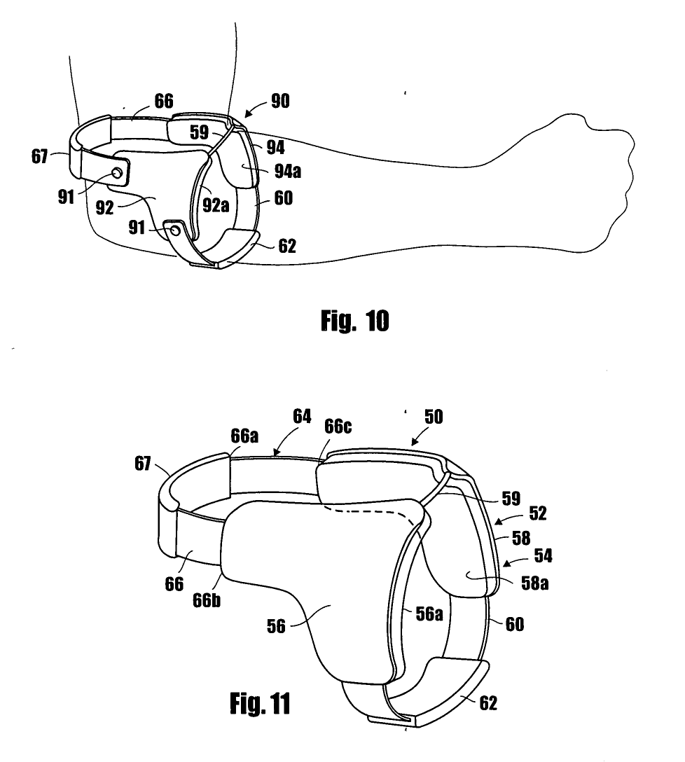

Turning now to FIG. 10, yet another form of the apparatus of the invention is there illustrated. The compression device of this embodiment of the invention, which is generally designated by the numeral 90, is somewhat similar to that illustrated in FIGS. 8 and 11 of the drawings and like numerals are used in FIG. 10 to identify like components. The primary difference between this latest form of the invention and that shown in FIGS. 8 and 11 resides in the fact that both strap 60 and strap 66 are pivotally interconnected by pivot pins 91 with the first and second spaced-apart side members 92 and 94 that are of similar construction to side members 76 and 78. As before, side members 92 and 94 are interconnected by adjustable connector member, or polymer cord 59, which is constructed and arranged so as to cause the spaced-apart arm-engaging side members to exert an inward pressure on selected areas of the forearm without substantially compressing other non-target areas and tissues. As illustrated in FIG. 10, when the device is in use, the side members 92 and 94 are positioned about the patient's forearm proximate the elbow so that the inelastic side members, which have concave inner skin-contacting surfaces 92a and 94a respectively, strategically overlie the forearm so as to enable effective transaxial counter-compression of the contact areas. With the construction shown in FIG. 10, straps 60 and 66 can be pivoted relative to the side members as may be required to comfortably fit the patient.

Device 90 can be used in the manner illustrated in FIG. 10 to effectively treat disorders related directly and indirectly to the patients forearm as, for example, treatment of the symptoms of arthritis, tendonitis, capsulities, carpal tunnel syndrome, lymphedema, trigger finger and ganglion cysts in the arm of a user, including symptoms related to the hand and fingers of the patient.

Having now described the invention in detail in accordance with the requirements of the patent statutes, those skilled in this art will have no difficulty in making changes and modifications in the individual parts or their relative assembly in order to meet specific requirements or conditions. Such changes and modifications may be made without departing from the scope and spirit of the invention, as set forth in the following claims.

I claim:

1. A method for treating the symptoms of biceps/triceps tendonitis and lymphedema in a user through the use of a generally circular device having two discrete compression plates projecting inwardly from the circular device and located at anatomically opposed locations along the circumference of the device, the method comprising the steps of:
(a) placing the device around the biceps and triceps portion of the user's arm to encircle said portion exclusively limiting autoposterior biceps and triceps muscular expansion during activity so that said discrete compression plates overlay respectively the biceps and triceps muscles in the arm; and
(b) drawing said compression plates toward one another to apply bipolar transaxial compression of equal magnitude simultaneously to discrete areas of the skin directly overlying said portion.

2. A method for treating the symptoms of arthritis, tendonitis, capsulities and ganglion cysts in the arm of a user through the use of a generally circular device having two discrete compression plates projecting inwardly from the circular device and located at anatomically opposed locations along the circumference of the device, the method comprising the steps of:
(a) placing the device around the elbow portion of the user's arm to encircle said portion so that said discrete compression plates overlie respectively the elbow portion of the arm; and
(b) drawing said compression plates toward one another to apply bipolar transaxial compression of equal magnitude simultaneously to discrete areas of the skin directly overlying said portion.

3. An elbow compressor device to be worn by a patient having a forearm, an elbow and a triceps, said device comprising:
(a) a first portion comprising a plurality of circumferentially spaced-apart forearm-engaging members for engaging selected portions of the forearm of the patient and a compression inducing member interconnecting said spaced-apart forearm-engaging members, said compression inducing member being so constructed and arranged as to cause said spaced-apart arm-engaging members to exert pressure on selected areas of the forearm without substantially compressing other non-target areas and tissues; and
(b) a second portion interconnected with said first portion, said second portion comprising a generally U-shaped flexible strap having a bite portion for engaging the patient's arm proximate the triceps and first and second end portions connected to said first portion.

4. The device as defined in claim 3, in which said compression inducing member comprises a first flexible strap.

5. The device as defined in claim 3, in which said compression inducing member comprises a polymer cord.

6. The device as defined in claim 3, in which said first and second end portions of said second flexible strap are pivotally connected to said first portion, said second flexible strap being movable between a first elbow-encircling position and a second position wherein said bite portion of said second strap is in close proximity with said first flexible strap.

7. The device as defined in claim 3 in which said spaced-apart forearm-engaging members are generally aspheric in shape.

8. The device as defined in claim 3, in which said first flexible strap of said first portion is adjustable to accommodate forearms of various sizes.

9. The device as defined in claim 3 in which said second strap further includes an arm-engaging member carried by said bite portion of said second strap.

10. A compressor device to be worn by a patient having a forearm, an elbow and a triceps, said device comprising:
(a) a first portion comprising a pair of spaced-apart forearm-engaging members for engaging selected portions of the forearm of the patient and a compression inducing member interconnecting said spaced-apart forearm-engaging members, said compression inducing member being so constructed and arranged as to cause said spaced-apart arm-engaging members to exert pressure on selected areas of the forearm without substantially compressing other non-target areas and tissues; and
(b) a second portion interconnected with said first portion, said second portion comprising a generally U-shaped stretchable strap having a bite portion for engaging the patient's arm proximate the triceps and first and second end portions connected to said first portion.

11. The device as defined in claim 10, in which said generally U-shaped stretchable strap is pivotally connected to said spaced-apart arm-engaging members.

12. The device as defined in claim 10, in which said compression inducing member comprises a polymer cord.

13. The device as defined in claim 12, in which said first portion further comprises a flexible strap, said strap being pivotally connected to said spaced-apart arm-engaging members.

14. The device as defined in claim 13 in which said strap further includes an arm-engaging member carried by said strap.

15. A method for treatment of the symptoms of arthritis, tendonitis, capsulities, carpal tunnel syndrome, lymphedema engages the patient's arm, trigger finger and ganglion cysts in the arm of a patient having a forearm, an elbow and a triceps through the use of a compression device having two spaced-apart forearm-engaging members, a compression inducing member interconnecting said spaced-apart forearm-engaging members for causing the spaced-apart arm-engaging members to exert pressure on the forearm and first and second straps connected to the spaced-apart forearm-engaging members, the method comprising the step of placing the compression device around the patient's arm so that the spaced-apart forearm-engaging members are located at anatomically opposed locations proximate the elbow, so that one of the first and second straps engages the patient's arm proximate the triceps and the other of the first and second straps engages the patient's forearm.
